(19) United States
(12) Patent Application Publication
(10) Pub. No.: US 2019/0365635 A1
(43) Pub. Date: Dec. 5, 2019

(54) Facial Hydrating Oil

(71) Applicant: Zugel Oil, LLC, Denver, CO (US)

(72) Inventor: Karen F. Wolfe, Denver, CO (US)

(21) Appl. No.: 16/424,695

(22) Filed: May 29, 2019

Related U.S. Application Data
(60) Provisional application No. 62/677,292, filed on May 29, 2018.

(51) Int. Cl. A61K 8/92; A61K 8/67; A61Q 17/04; A61Q 19/00

(52) U.S. Cl. CPC A61K 8/922; A61K 8/678; A61K 2800/522; A61Q 19/00; A61Q 17/04

(57) ABSTRACT

Facial hydrating oil compositions comprise sweet almond seed oil, carrot seed oil, rose hip seed oil, and a tocopherol. Such compositions are useful as cosmetics for hydrating skin and minimizing formation of fine lines and wrinkles.

### CROSS-REFERENCE TO RELATED APPLICATION

This application is a nonprovisional of and claims the benefit under 35 U.S.C. 119(e) of U.S. Provisional Patent Application No. 62/677,292, filed May 29, 2018, the contents of which are incorporated herein by reference as if fully disclosed herein.

### TECHNICAL FIELD

Provided herein are cosmetic hydrating compositions. More particularly, the cosmetic hydrating compositions contain sweet almond oil for facial application and hydration.

### BACKGROUND OF THE INVENTION

Facial creams and facial oils are useful for protecting skin from harmful pollutants, sun and the weather, including colder and hotter temperatures, wind, and dry air. Facial creams and facial oils are typically used to moisturize the skin, though ideally such skin care products would also nourish the skin with nutrients and antioxidants helpful in minimizing wrinkles and sunspots, promote softness, and aid in the healing of minor skin injuries.

Unfortunately, many creams and oils are not well absorbed into the skin, and leave lingering oil or residue on the face. Many creams and oils also only moisturize and do not provide added benefits. As such, users typically apply two or more facial cosmetics at a time to obtain maximum benefits.

The present embodiments are directed toward overcoming one or more of the problems discussed above.

### SUMMARY OF THE INVENTION

Various features and advantages of the embodiments will be set forth in part in the following description, and may be obvious from the description, or may be learned from practice of the embodiments.

Provided herein is a cosmetic composition comprising sweet almond oil, carrot seed oil, rose hip seed oil, and a tocopherol. In some embodiments, one or more of the ingredients are organic, for example, organic sweet almond oil, organic carrot seed oil, organic rose hip seed oil, or organic vitamin E. In some embodiments, one or more of the ingredients are natural, for example, natural sweet almond oil, natural carrot seed oil, natural rose hip seed oil, or natural vitamin E. In some embodiments, one or more of the sweet almond oil, carrot seed oil, the rose hip seed oil, or the tocopherol are derived from natural plant sources.

In some embodiments, the tocopherol is vitamin E.

The beneficial characteristics of the compositions provided are the combined moisturizing effects with other benefits including reducing the appearance of wrinkles, calming redness, increasing skin softness, and aiding in healing. In addition, the compositions absorb well with no residue.

In some embodiments, the sweet almond oil is present in an amount of about 98% by volume. In some embodiments, the carrot seed oil is present in an amount of less than about 1% by volume. In some embodiments, the rose hip seed oil is present in an amount of less than about 1%. In some embodiments, the tocopherol is present in an amount of less than about 0.1%. In some embodiments, the tocopherol is an antioxidant.

Also provided herein is a cosmetic composition consisting essentially of sweet almond oil, carrot seed oil, rose hip seed oil, and a tocopherol. In embodiments, the composition may also comprise trace amounts of at least one or more of fragrances or sunscreens. The trace amounts may be less than about 0.03%.

These and other features, aspects and advantages of the present embodiments will become better understood with reference to the description, examples, and appended claims.

### DESCRIPTION

Provided herein are cosmetic compositions comprising sweet almond oil, such as organic sweet almond oil. These compositions are useful for protecting skin from the environment, including colder temperatures, hotter temperatures, harmful chemicals, wind, and dry air, nourishing the skin by providing nutrients and antioxidants helpful in minimizing wrinkles and sunspots, promoting softness, calming redness and aiding in the healing of minor skin injuries and/or blemishes. In addition, the compositions are absorbed quickly without residue.

In addition to the sweet almond oil, the cosmetic compositions may further comprise carrot seed oil, rose hip seed oil, and tocopherol. The combination of the four ingredients provides a unique and extremely effective product. The compositions provided herein surprisingly combine the properties of long lasting and effective hydration, aiding in the reduction of sun damage, aiding in the reduction of the appearances of fine lines, softening the skin, calming redness, and aiding in healing skin all in one formulation. Such product eliminates the need for application of multiple products in order to obtain the same benefits. For example, the elimination of one component, the tocopherol (vitamin E), from the composition, results in a reduced length of time of skin hydration.

Sweet almond oil, or prunus amygdalus oil, is a glyceryl oleate obtained from the dried kernel of sweet almonds. The oil is colorless and almost odorless, and is well tolerated by the skin. Sweet almond oil is useful herein as a carrier oil as it is easily absorbed and protects the skin from its environment. Additional benefits of sweet almond oil include that it does not clog pores, it is an antioxidant, and it absorbs free radicals generated in the skin by exposure to UV rays from the sun, and thus repairs sun damage. The texture of sweet almond oil allows makeup and sunscreen to be effectively and positively applied directly after application, while other oils do not work as well.

In some embodiments, the sweet almond seed oil is provided in an amount of about 95% to about 99% by volume, about 96% to about 99%, or about 98% by volume, or about 99% by volume, or about 97% by volume.

The composition provided herein further comprises carrot seed oil. Carrot seed oil is included, in some aspects, for its antioxidant properties. Carrot seed oil contains carotene and vitamin A, which purports to protect skin from wrinkles and diminished elasticity. Carrot seed oil has a viscous consistency, a yellowish brown color, and a woody, earthy, and root-like fragrance. Carrot seed oil is a natural sunscreen and promotes cell regeneration. Unlike other oils, carrot seed oil combines surprisingly well with sweet almond oil and rose hip seed oil.

In some embodiments, the carrot seed oil is provided in an amount of less than about 1.00% by volume. In some embodiments, the carrot seed oil is provided in an amount of about 0.8% to about 0.9% by volume, or about 0.80% to about 0.85% by volume, or about 0.83% by volume.

The compositions provided herein further comprise rose hip seed oil. Rose hip seed oil is included, in some aspects, as it absorbs into the skin quickly and does not leave an oily residue. Its emollient properties are also desirable, in addition to its beneficial effects on cell regeneration and its antioxidant properties.

In some embodiments, the rose hip seed oil is provided in an amount of less than about 1.00% by volume. In some embodiments, the rose hip seed oil is provided in an amount of about 0.75% to about 0.85% by volume, or about 0.78% to about 0.82%, or about 0.80% by volume.

The compositions provided herein further comprise a tocopherol. An illustrative tocopherol is vitamin E. In some aspects, the composition comprises vitamin E. Tocopherol is an antioxidant, and in some aspects improves the feel of the oil product. In addition, tocopherol increases the duration of the hydration of the composition. The tocopherol may be derived from an organic source. In embodiments, the tocopherol comprises at least one of alpha-tocopherol, beta-tocopherol, gamma-tocopherol, or delta-tocopherol.

In some embodiments, the tocopherol is provided in an amount of less than about 0.05% by volume, or less than about 0.04%, or about 0.03% by volume.

It is contemplated herein that additional ingredients can be added to the base formulation of sweet almond seed oil, carrot seed oil, rose hip seed oil, and tocopherol. For example, additional components include essential oils, such as those used for fragrances or other properties, and sunscreens. In embodiments, the sunscreens may be added in amounts from about 3% to about 20% by volume. In embodiments, the fragrances and/or the sunscreens may be added in trace amounts. In embodiments, trace amounts may be defined as less than about 0.03%, or less than about 0.02%, or about 0.01% by volume.

The compositions provided herein can be used as cosmetics, for example, for application to an individual's skin. It is contemplated that the composition is useful for both men and women. The composition is formulated for application to the face and neck, but it is contemplated herein that the composition would also be useful on any skin, for example, the hands and arms or feet, and any skin type, including oily or dry skin. Also provided herein are methods of slowing the formation of fine lines and wrinkles on the neck and face. In some aspects, the method comprises application of a small amount of the composition to fingertips, then gently massaging the composition onto the face and neck in a uniform manner.

Also provided herein are methods of aiding in reducing the appearance of rosacea. In some aspects, the method comprises application of a small amount of the composition to the area of skin affected with rosacea.

Also provided herein are methods of providing lasting moisturizer to the skin. In some aspects, the method comprises application of an amount of the composition to the area of the skin in need of moisturizer.

Also provided are methods for rehydrating skin following use of a face serum. In some aspects, the method comprises application of an amount of the composition to the area of the skin in need of rehydration. In some embodiments, the face serum comprises Retin A and/or more generally a retinol.

Also provided are methods for minimizing peeling of the skin following use of a face serum. In some aspects, the method comprises application of an amount of the composition to the area of the skin on which the face serum was applied. In some embodiments, the face serum comprises Retin A and/or more generally a retinol.

While the embodiments have particularly shown and described with reference to a number of embodiments, it would be understood by those skilled in the art that changes in the form and details may be made to the various embodiments disclosed herein without departing from the spirit and scope of the embodiments and that the various embodiments disclosed herein are not intended to act as limitations on the scope of the claims.

### EXAMPLES

The following examples are provided for illustrative purposes only and are not intended to limit the scope of the embodiments.

### Example 1

An exemplary composition was formulated as follows:

Formula for 30 ml bottle:
29.5 ml -Organic prunus amygdalus (sweet almond) oil, about 98.34%
0.25 ml—Organic Daucus Carota (carrot) seed oil, about 0.83%
0.24 ml—Organic Rosa Mosqueta (rose hip) seed oil, about 0.80%
0.01 ml—Tocopherol (Vitamin E), about 0.03%

The ingredients were added sequentially, and shaken to ensure even distribution of all the ingredients.

### Example 2

In this example, the facial oil composition as provided in Example 1 was tested with 65 women and 5 men volunteers. Most volunteers reported that their skin was softer in 3-7 days. In addition, most felt that the product hydrated their skin throughout the day. Those volunteers who suffered from rosacea all noticed that the product calmed the redness from their skin. Eighty five percent of the volunteers loved or liked the product and wanted to purchase the product. Those who had oily skin noticed a decrease in natural oil secretion and some noticed a reduction in acne.

What is claimed is:

1. A cosmetic composition comprising sweet almond oil, carrot seed oil, rose hip seed oil, and tocopherol.

2. The composition of claim 1, wherein the tocopherol is an antioxidant.

3. The composition of claim 1, wherein the sweet almond oil is present in an amount of about 98%.

4. The composition of claim 1, wherein the carrot seed oil is present in an amount of less than about 1%.

5. The composition of claim 1, wherein the rose hip seed oil is present in an amount of less than about 1%.

6. The composition of claim 1, wherein the tocopherol is present in an amount of less than about 0.1%.

7. The composition of claim 1, wherein the sweet almond oil is present in an amount of about 98%, the carrot seed oil is present in an amount of less than about 1%, the rose hip seed oil is present in an amount less than about 1%, and the tocopherol is present in an amount less than about 0.1%.

8. The composition of claim 1, wherein the tocopherol is Vitamin E.

9. The composition of claim 8, wherein the tocopherol comprises at least one of alpha-tocopherol, beta-tocopherol, gamma-tocopherol, or delta-tocopherol.

10. The composition of claim 1, wherein one or more of the sweet almond oil, carrot seed oil, the rose hip seed oil, or the tocopherol are derived from natural plant sources.

11. The composition of claim 1, further comprising one or more sunscreens.

12. A facial hydrating oil composition consisting essentially of sweet almond oil, carrot seed oil, rose hip seed oil, and a tocopherol.

13. The composition of claim 12, further comprising trace amounts of at least one or more of fragrances or sunscreens.

14. The composition of claim 13, wherein the trace amounts are less than about 0.03%.

15. A facial cream comprising a mixture of a sweet almond oil, a carrot seed oil, a rose hip seed oil, and a vitamin E oil.

16. The mixture of claim 15, wherein the mixture further comprises a sunscreen.

17. The mixture of claim 16, wherein the sunscreen is present in an amount of between about 3% to about 20% by volume.

18. The mixture of claim 15, wherein the mixture further comprises a fragrance.

19. The mixture of claim 18, wherein the fragrance is an essential oil.
